(19) United States
(12) Patent Application Publication
(10) Pub. No.: US 2003/0220678 A1
(43) Pub. Date: Nov. 27, 2003

(54) Adjustable implantable captivation fixation anchor-stop

(76) Inventors: Carole A. Tronnes, St. Paul, MN (US); Keith Carlton, Minneapolis, MN (US); Martin T. Gerber, Maple Grove, MN (US); Daniel J. Stetson, Lino Lakes, MN (US); John M. Swoyer, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/152,555

(22) Filed: May 21, 2002

(51) Int. Cl. A61N 1/05

(52) U.S. Cl. 607/133; 607/119

(57) ABSTRACT

An implantable medical or intramuscular lead system, such as for use as a gastric lead, and method of use in which electrodes along the lead are imbedded in tissue. The system includes a clip having two arms biased to a closed position clamping the lead. The arms of the clip are movable against the bias to an open position allowing the clip to be moved along the length of the lead, or the clip to be attached or removed in the lateral direction. The system facilitates implantation of the lead in tissue, and may be particularly suited for minimally invasive implantation, such as laparoscopically.

### RELATED APPLICATION

This disclosure is related to the following co-pending application entitled “IMPLANTABLE MEDICAL DEVICE WITH CAPTIVATION FIXATION” by Carole Tronnes, John M. Swoyer and Martin T. Gerber (application Ser. No. 10/121,484; filed Apr. 12, 2002), which is not admitted as prior art with respect to the present disclosure by its mention in this section.

### FIELD OF THE INVENTION

This invention relates to systems and methods for anchoring a medical lead or therapy delivery device to tissue.

### BACKGROUND OF THE INVENTION

The human body GI tract comprises the esophagus, the stomach, the small intestine, the large intestine, the colon, and the anal sphincter and is generally described as having a tract axis. Like other organs of the body, most notably the heart, these organs naturally undergo regular rhythmic contractions. In particular these contractions take the form of peristaltic contractions and are essential for the movement of food through each of the respective organs. Like the heart, these contractions are the result of regular rhythmic electrical depolarizations of the underlying tissue. With regards to the small intestine and large intestine, normal electrical depolarizations (“slow waves”) typically occur at a rate of approximately 15 and 1 beats per minute (bpm) respectively. Similarly, in the stomach, normal slow waves typically occur at a rate approximately 3 bpm. Not all of these depolarizations, however, normally result in a contraction of the organ. Rather contractions occur upon the occurrence of a normal electrical depolarizations followed by a series of high frequency spike activity.

In some individuals, however, either the regular rhythmic peristaltic contractions do not occur or the regular rhythmic electrical depolarizations do not occur or both do not occur. In each of these situations the movement of food may be seriously inhibited or even disabled. Such a condition is often called “gastroparesis” when it occurs in the stomach 30. Gastroparesis is a chronic gastric motility disorder in which there is delayed gastric emptying of solids or liquids or both Symptoms of gastroparesis may range from early satiety and nausea in mild cases to chronic vomiting, dehydration, and nutritional compromise in severe cases. Similar motility disorders occur in the other organs of the GI tract, although by different names.

Diagnosis of gastroparesis is based on demonstration of delayed gastric emptying of a radiolabeled solid meal in the absence of mechanical obstruction. Gastroparesis may occur for a number of reasons. Approximately one third of patients with gastroparesis, however, have no identifiable underlying cause (often called idiopathic gastroparesis). Management of gastroparesis involves four areas: (1) prokinetic drugs, (2) antiemetic drugs, (3) nutritional support, and (4) surgical therapy (in a very small subset of patients.) Gastroparesis is often a chronic, relapsing condition; 80% of patients require maintenance antiemetic and prokinetic therapy and 20% require long-term nutritional supplementation. Other maladies such as tachygastria or bradygastria can also hinder coordinated muscular motor activity of the GI tract, possibly resulting in either stasis or nausea or vomiting or a combination thereof.

The undesired effect of these conditions is a reduced ability or complete failure to efficiently propel intestinal contents down the digestive tract. This results in malassimilation of liquid or food by the absorbing mucosa of the intestinal tract. If this condition is not corrected, malnutrition or even starvation may occur. Moreover nausea or vomiting or both may also occur. Whereas some of these disease states can be corrected by medication or by simple surgery, in most cases treatment with drugs is not adequately effective, and surgery often has intolerable physiologic effects on the body.

For many years, sensing of the peristaltic electrical wave and gastrointestinal stimulation at various sites on or in the GI tract wall of the digestive system or nerves associated therewith have been conducted to diagnose and treat these various conditions. Examples sensing and GI tract stimulation are set forth in commonly assigned U.S. Pat. Nos. 5,507,289, 6,026,326, and 6,216,039, all of which are incorporated herein by reference.

Electrical stimuli are applied from the neurostimulator implantable pulse generator (IPG 50) through leads and electrodes affixed at sites in the body of the patient or the GI tract wall that permit the electrical stimulus to produce a local contraction of a desired portion of the GI tract. The sites of the GI tract wall comprise the outermost serosa or sub-serosally in the inner, circumferential and longitudinal (and oblique in the case of the stomach) smooth muscle layers referred to as the “muscularis externa”. The smooth muscle is preferably comprised of innervated muscle tissue, and it is theorized that the smooth muscle is neurally electrically stimulated through the nerves associated with and innervating the muscle tissue in order to produce the contraction of the smooth muscle.

An implantable method and system for electrical stimulation of smooth muscle with intact local gastric nerves comprising a portion of the GI tract is disclosed in the '607 patent. The electrical stimulation of the smooth muscle effects local contractions at sites of a portion of the GI tract that are artificially propagated distally therethrough in order to facilitate or aid at least a partial emptying of such portion. This stimulation attempts to create a simulated system that reproduces the spatial and temporal organization of normal gastric electrical activity by creating and controlling local circumferential non-propagated contractions. In this simulated gastric pacing system, each local circumferential contraction is invoked by applying an electrical stimulus to the smooth muscle circumferentially about the portion of the GI tract in a plane substantially perpendicular to the longitudinal axis of the portion. The electrical stimulus is applied at a proximal location and at at least one distal location. The distal location is in axially spaced relationship relative to the proximal location. Further, the applied electrical stimulus is selected to be sufficient to stimulate the smooth muscle to produce the local circumferential contractions at the proximal and distal locations.

The Medtronic® Itrel III Model 7425 IPG and pairs of the unipolar Model 4300 or Model 4301 or Model 4351 “single pass” leads available from MEDTRONIC, INC., Minneapolis, Minn., have been implanted to provide stimulation to sites in the stomach wall to treat chronic nausea and vomiting associated with gastroparesis. The unipolar electrode of these leads comprises a length of exposed lead conductor and is of the type disclosed in commonly assigned U.S. Pat. Nos. 5,425,751, 5,716,392 and 5,861,014, which are incorporated herein by reference. The above-referenced '039 patent and the '014 patent disclose the Model 4300 lead sewn through the serosa laterally into the muscularis externa to dispose the stimulation/sense electrode therein. A large incision is necessary to access the site, and a needle is used to perforate the serosa and muscularis externa laterally without fully penetrating the wall and to draw the stimulation/sense electrode into the muscularis externa. A laparoscopic approach can be taken, but it is difficult to fixate the lead at the implant site.

The stimulation/sense electrodes conventionally employed in such gastrointestinal stimulation systems are formed of bio-compatible material shaped to either bear against the serosa or penetrate sub-serosally into the muscularis externa and polished to present an impervious outer surface. It is also suggested in the above-referenced '014 patent that the exposed electrode(s) of the single pass lead can alternatively be formed of other biocompatible electrode materials, including porous, platinized structures and could feature various pharmaceutical agents. Suggested pharmaceutical agents include dexamethasone sodium phosphate or beclomethasone phosphate in order to minimize the inflammatory response of the tissue to the implanted lead.

When the stimulation leads are inserted or implanted, they are typically anchored in place by sewing the lead through the serosa laterally into the muscularis externa at both the proximal lead entrance site as well as the distal end of the electrode(s). The anchoring is important for the insertion or implantation procedure because this is intended to prevent the stimulation lead from migrating away from a specifically selected stimulation site. The anchoring process is often used during surgical procedures where there is limited space to anchor and secure the lead to tissue, and time constraints to complete the procedure rapidly. For some procedures, anchor the lead to the stomach wall can be one of the most time consuming and invasive portions of the stimulation lead insertion procedure. Clinicians inserting and anchoring therapy delivery elements typically prefer to perform the procedure rapidly, in a minimally invasive manner, and fix the therapy delivery element in a manner that reduces the opportunity for the therapy delivery element to migrate if practicable. Previous stimulation lead anchoring systems can have one or more of the following limitations along with other limitations such as being difficult to use for minimally invasive procedures, difficult to secure the simulation lead in the desired position and susceptibility to lead migration.

### BRIEF SUMMARY OF THE INVENTION

The invention provides a system and method for anchoring therapy delivery devices and medical leads, such as gastric leads, in tissue. Preferred embodiments of this invention facilitate, among other things, minimally invasive procedures, reliably securing the therapy delivery element or electrodes in position within tissue, and rapid placement to reduce procedure time. The therapy delivery element may be embodied in a tissue stimulation lead adapted to be implanted within the body at a site to conduct electrical stimulation from an implantable or external neurostimulator to the site and to conduct electrical signals the site to the implantable or external neurostimulator.

Exemplary embodiments of the invention pertain to gastrointestinal leads, which are adapted to be implanted within the body at a site of the gastrointestinal tract (GI tract) to conduct electrical stimulation from an implantable or external electrical neurostimulator to the site, and/or to conduct electrical signals of the GI tract from the site to the implantable or external electrical neurostimulator.

In a first embodiment of the invetion, a medical lead system generally comprises a lead having a length defining longitudinal and lateral directions and at least one electrode, and a clip having two arms biased to a closed position clamping the lead. The arms are movable against the bias to an open position allowing the clip to be moved along the length of the lead, or the clip to be attached or removed in the lateral direction.

In a second aspect of the invention, an implantable therapy delivery system generally comprises an implantable therapy delivery device; at least one elongate therapy delivery element coupled to the implantable therapy delivery device, and an adjustable anchor coupleable to the therapy delivery element. The therapy delivery element having a length defining longitudinal and lateral directions. The adjustable anchor is implantable and includes a grip element, and at least two extension elements connected to the therapy grip element. The grip element is configured to be actuated between: (a) an open position in which the anchor may be attached or removed from the therapy delivery element laterally, or repositioned along the length of the therapy delivery element; and (b) a closed position in which the grip element grips the therapy delivery element. The extension element extends substantially perpendicular from the therapy delivery element, and the extension elements being configured to actuate the therapy grip element.

Preferably, the anchor may be removed or attached at any point along the length of the therapy delivery element when the grip element is in its open position.

Also, preferably, the open position includes: a first open position in which repositioned along the length of the therapy delivery element; and a second open position further open than the first open position in which the anchor may be attached or removed from the therapy delivery element laterally.

In addition, a means is preferably provided for indicating that the grip element is in each of its first and second open positions. For example, the means for indicating that the grip element is in each of its first and second open positions may comprises a tool for manipulating the anchor and indicating whether the grip element is in each of its first and second open positions, or a detent for releasably retaining the grip element in either its first or second open positions.

Most preferably, the grip element comprises two jaws for clamping the therapy delivery element, the jaws having roughed, toothed, grooved, or sticky surfaces for gripping the therapy delivery element. For example, the jaws may include interlocking teeth or grooves.

In various embodiments of the invention, the grip element may include two jaws for clamping the therapy delivery element, with the jaws each having a free end and including cooperable alignment means for aligning the free ends of each jaws relative to one another when the grip element is in the closed position, thereby tending to prevent skewing of the jaws when the jaws are in the closed position. For example, the cooperable alignment means may comprise complementary interlocking structures on the free ends of the jaws that are brought into interlocking relationship when the jaws are brought to the closed position Examples of complementary interlocking structures include complementary projecting and recessed portions on each jaw such that the projecting and recessed portions of one jaw define an opposite or negative structure compared to the projecting and recessed portions of the other jaw.

In fourth embodiment of the invention, an implantable medical lead system generally comprises at least one elongate lead having a length and opposite ends; a first stop mounted on the lead; a second stop mounted on the lead for movement along the length of the lead relative to the first stop to capture the tissue between the stops so that the lead is retained in position; and a clip having two arms biased to a closed position clamping the lead to prevent the second stop from moving. The arms of the clip are movable against the bias to an open position allowing the clip to be moved along the length of the lead to release the second stop.

In a fifth embodiment of the invention, a medical lead system generally comprises a lead having a length defining longitudinal and lateral directions and at least one electrode; and a clip formed of spring wire to have two arms biased to a closed position clamping the lead. The arms are movable against the bias to an open position allowing the clip to be moved along the length of the lead, or the clip to be attached or removed in the lateral direction, the arms being offset in the longitudinal direction relative to one another.

Preferably, in the fifth embodiment of the invention, the spring wire is bent to form a helical hinge section connecting the two arms and permitting movement of the arms between the open position and a closed position. Also, preferably, the spring wire is bent to form an arcuate section along each arm for engaging the medical lead, with the arms crossing over each other between the hinge and the arcuate sections. The arcuate sections each define a concave side engaging the medical lead.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is an illustration of a patient with a gastric stimulation system implanted;

FIG. 2 is an illustration of a gastric lead implanted in gastric tissue;

FIG. 3A is a side view of a gastric stimulation lead with an insertion needle;

FIG. 3B is an enlarged view of portion 3B of FIG. 3A;

FIG. 4 is a flow chart of a method of implantation of a gastric lead;

FIG. 5A is perspective view of a first embodiment of the anchor clip;

FIG. 5B is a front view of a second embodiment of anchor clip;

FIG. 5C is a partial, frontal view of an application tool for use with various embodiments of the anchor clip;

FIG. 6 is a perspective view of a third embodiment of the anchor clip;

FIGS. 7A and 7B are perspective and side elevational views of an anchor clip and tissue interface feature;

FIGS. 8A-8D are top, side, front and perspective views of a three-piece adjustable anchor embodiment;

FIG. 9 is an illustration of a three-piece adjustable anchor and tissue interface feature in use;

FIG. 10 is an illustration of a three-piece adjustable anchor and winged tissue interface feature in use;

FIG. 11A is an exploded view of an alternative adjustable anchor-stop; and

FIGS. 11B and 11C are side views of the alternative adjustable anchor-stop illustrating open and closed positions respectively.

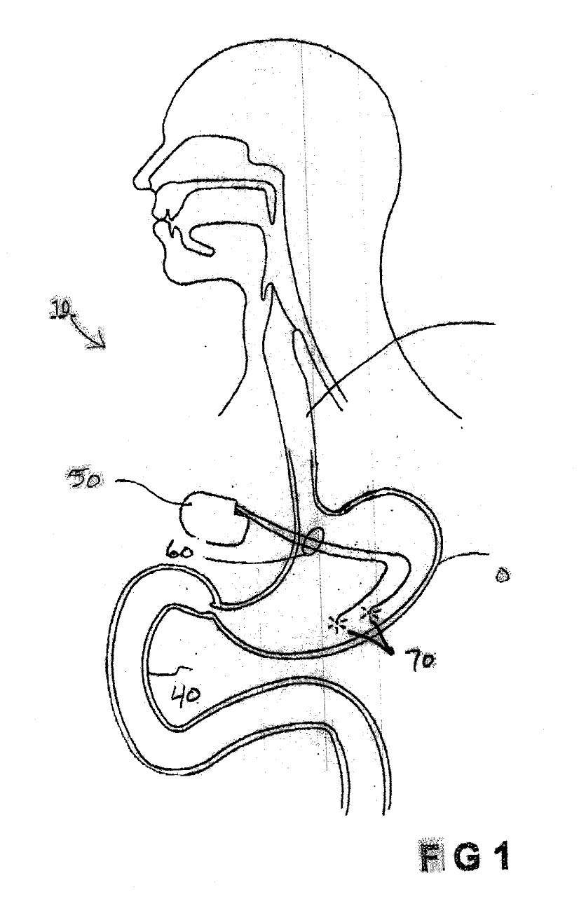

### DETAILED DESCRIPTION OF THE INVENTION

The medical lead system, intramuscular leads and methods of attachment system of the present invention provides the surgeon with more options for therapy delivery element placement within tissue. This invention can be used wherever it is desirable to sense or deliver a therapy to tissue. Examples of applicable areas of application include but are not limited to tissue stimulation including muscular stimulation such as GI tract stimulation and including muscle stimulation used in dynamic graciloplasty.

Embodiments of the invention are ideally suited for a gastric stimulation application or other tissue stimulation applications. Various embodiments are particularly useful where it is desirable to impinge a muscle with a stimulation electrode and to captivate the lead within the muscle. Preferred embodiments are amenable to a quick placement and anchoring through a cannula as commonly used in laparoscopic procedures and other types of minimally invasive surgical techniques.

The IPG 50 can comprise a hermetically enclosed implantable pulse generator (IPG), which includes a battery and an electrical operating system powered by a battery. The IPG 50 operating system can sense the gastro-electrical signals conducted through the electrodes 70, and pulse generator circuitry that generates electrical stimulation pulses that are conducted through the electrodes 70 to the stomach 30 in accordance with a programmed operating mode and programmed operating parameter values. It will be understood that the stimulation/sense electrodes 70 can all function as sensing and stimulation electrodes, and the selection of the stimulation/sense electrodes 70 for sensing and stimulation functions can be programmed into the IPG 50.

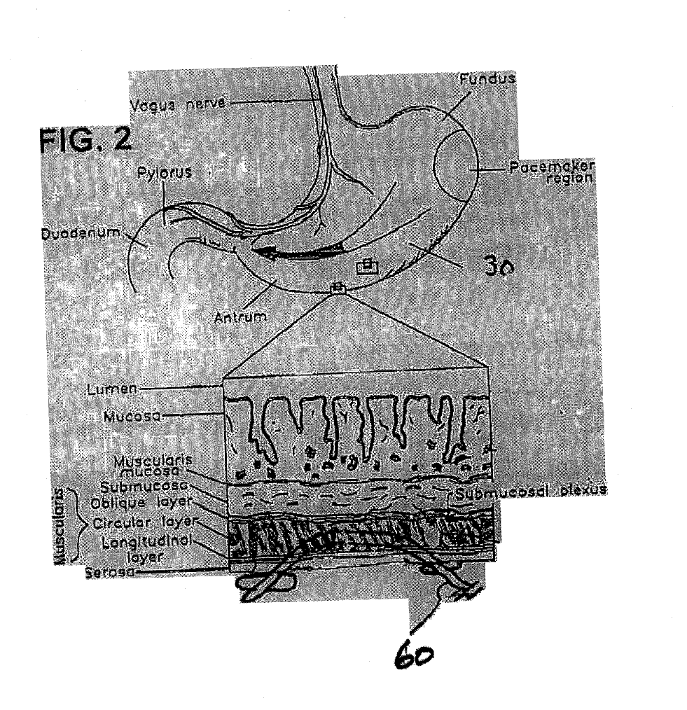

The stomach wall of the stomach 30 comprises essentially seven layers of tissue that are shown in cross-section in FIG. 2. The seven tissue layers include the oblique, circular, and longitudinal muscle layers of the muscularis externa that contract and expand as described above, interposed between the interior stomach mucosa and the external serosa. In the preferred embodiments, the intramuscular lead in FIG. 3 is drawn through the muscle using the integral needle 80 to perforate the serosa and lodge in the electrodes 70 in the muscularis externa, particularly within the thickest circular layer as shown in FIGS. 2. The typical depth of penetration of the electrodes 70 is preferably in the range of 1 mm to 15 mm when the site comprises the antrum or in the range of 1 mm to 10 mm when the site comprises corpus or fundus to ensure that the electrodes 70 does not extend substantially through the stomach wall.

FIG. 3 shows a stimulation lead 60 embodiment. The implantable stimulation lead 60 configured for laparoscopic implantation has a lead body 90, at least one electrode 70 (e.g., at least two electrodes for a bipolar configuration), at least one connector 80, and at least one conductor. The lead body 90 has a distal body end, a proximal body end. The electrode(s) 70 is coupled to the distal body end, and the connector 80 is coupled to the proximal body end. There is a conductor carried in the lead body 90 to electrically connect the electrode 70 to the connector 80. The conductor is insulated by the lead body 90. The implantable stimulation lead 60 having one or more isolated electrodes 70, having a diameter of approximately a 0.127 cm (0.050 inch), having an anchor-stop fixed to the lead body 90 proximal to the electrodes 70 to act as a proximal stop 100 and having a suture wire 110 and needle 80 attached to the end of the lead to assist in the introduction of the lead into tissue.

The embodiment shown in FIG. 3 is implanted by utilizing the needle 80 to size the amount of tissue to be captivated between the anchor-stops. The length of the needle 80 is sized to perform the function of as a gauge so the physician can obtain optimal electrode placement. The needle-gauge 80 is used to obtain appropriate insertion depth and to obtain the appropriate amount of tissue to be captivated for stimulation by the electrodes 70. The diameter of the needle 80 is chosen to allow the lead body 90 to pass through the channel created by the needle 80 without difficulty. The length of the needle 80 is determined by the length of electrode and lead to be imbedded within the tissue along with an additional length to allow manipulation of the needle 80 with the appropriate tool. The shape of the needle 80 is determined by ergonomics and by the need to allow the passage of the lead down a small cannula.

The anchor-stop 100 can be permanently attached to the lead body 90. An alternative embodiment is an anchor that can be sutured or can be permanently fixed to the lead body 90 by other means by the physician. This would be desirable when variability of the tissue stimulation application does not allow a consistent placement of the anchor. When the anchor 100 is permanently pre-attached to the lead body 90, the anchor is attached at a distance away from the electrode 70 closest to the exit site to prevent inadvertent stimulation of adjacent bodily fluids or tissue. In a muscle stimulation application, this distance is typically 5 mm but may vary depending on application.

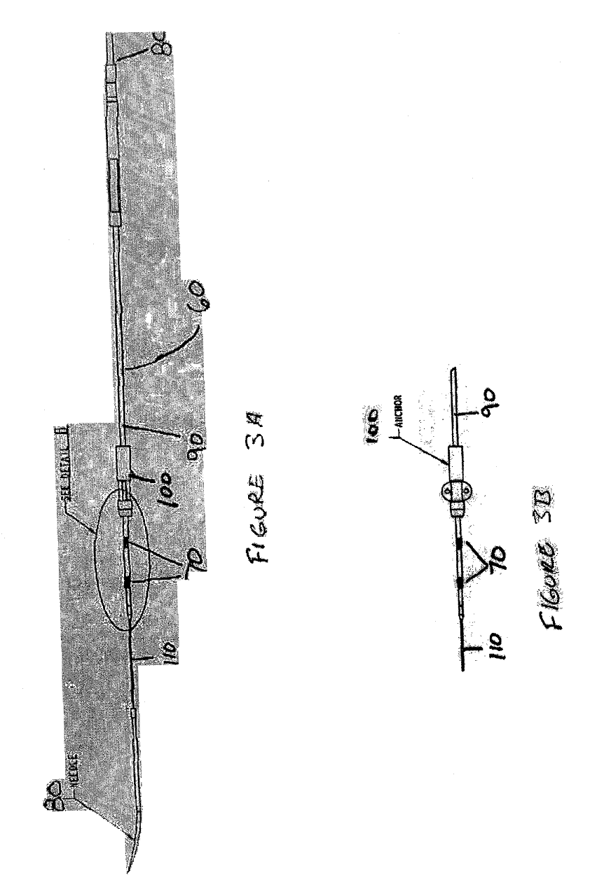

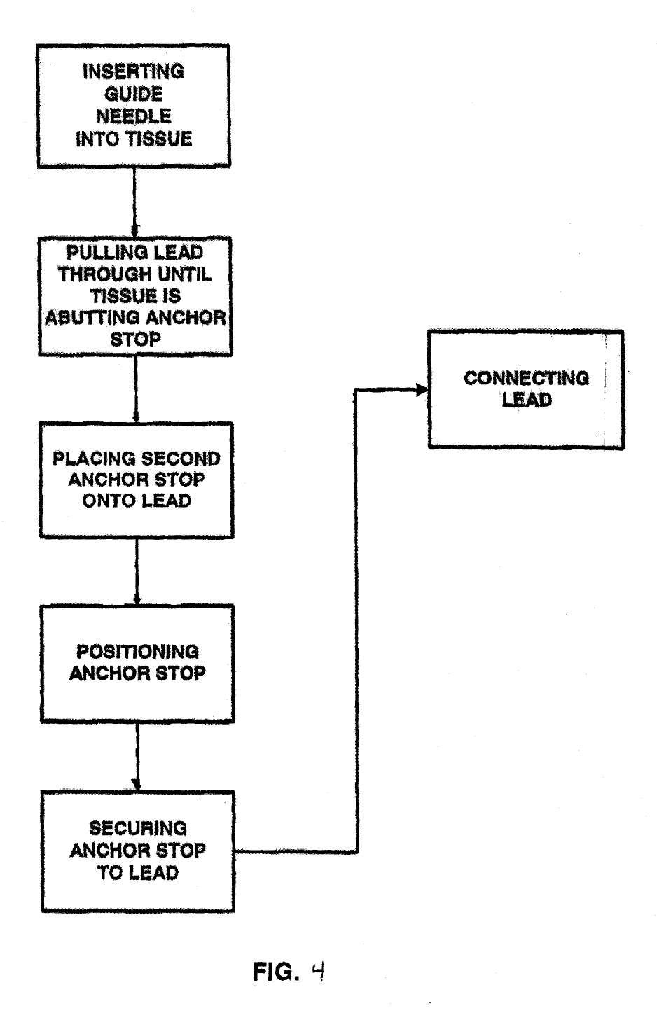

The method for implantation of the implantable medical device with captivation fixation is shown in FIG. 4. First, the lead 60 is inserted into the target tissue using the guide needle 80 as a gauge to aid in the placement of the electrode(s) 70. Next, the lead body 90 is pulled through the tissue until the anchor stop 100 is abutting tissue adjacent to the targeted stimulation site. Next, a second anchor stop 100 is placed on the lead body 90 and positioned on the lead adjacent on the tissue surface adjacent to the targeted stimulation site and opposite the first anchor stop 100. Next, the second anchor stop 100 is secured to the lead and the lead is connected to the IPG 50.

The adjustable anchor-stop 120 is inserted on to the lead 60 after the lead 60 has been implanted into the tissue and drawn to the first anchor-stop 100. The adjustable anchor-stop 120 is then advanced onto the lead in a location approximate to the desired location abutting the tissue between the fixed and the adjustable anchor-stop 120. The anchor stop is biased to an open position so that it can be inserted onto the lead 60 in a lateral direction; thus simplifying the lead anchoring by avoiding the need to thread the anchor on the distal or proximal end of the lead 60. The adjustable anchor is then biased to the closed position where it grips the lead 60 and captivates the tissue to be stimulated between the anchors.

The adjustable anchor clip or anchor-stop can take different forms. In one configuration as shown in FIGS. 5A and 5B, the basic design consists of spring wire or metal that has been formed into a shape with a coiled or helical spring 130 at one end to provide the holding force of the two arms or jaws 140. The jaws are shaped in a manner to captivate and grip the lead 60. The two jaws 140 biased to a closed position clamping the lead. The jaws 140 are movable against the bias to an open position allowing the clip to be moved along the length of the lead, or the clip to be attached or removed in the lateral direction. The jaws 140 may be offset in the longitudinal direction (of the lead) relative to one another. The adjustable anchor-stop 120 is formed out of a spring metal wire such as stainless steel or nitinol to allow the repeated opening and closing the anchor-stop may see when it is being positioned on the lead body.

To facilitate easy placement during a minimally invasive operation, an application tool 145 such as the one shown in FIG. 5C could be used to bias the anchor-stop in an open position until the anchor-stop is placed in its final position. The application tool 145 includes a fixed jaw 147 and a movable jaw 149. The movable jaw 149 of the application tool 145 squeezes the anchor clip or stop to open the anchor clip as the movable jaw 149 is moved from its open position toward its closed position. The movable jaw 149 may be moved between its open and closed position via a longitudinally extending actuating mechanism, allowing the jaw to be operated from the other end of the tool. This may be advantageous, for example, if the other end of the application tool is outside the body and the anchor clip is being placed laporascopically.

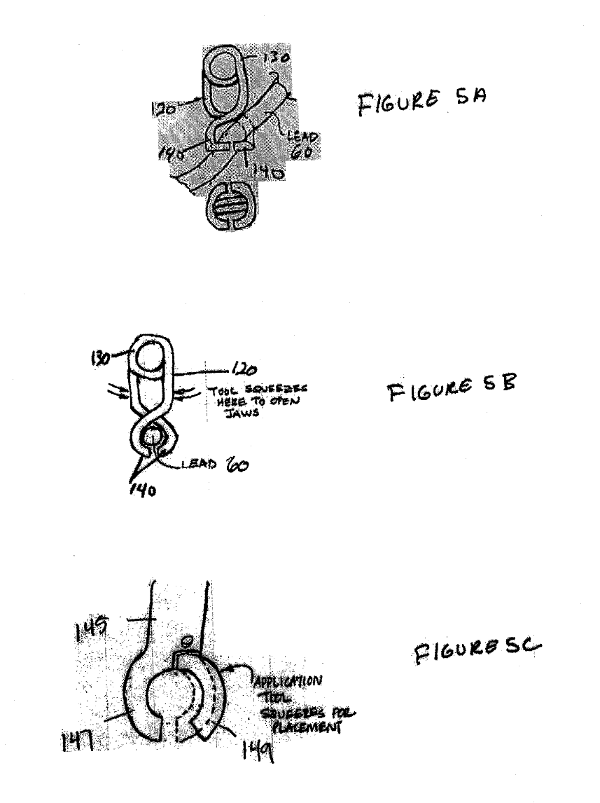

Alternatively, the anchor-stop (FIG. 6) could take a form where the jaws or arms of the adjustable anchor-stop 120 have opposing semicircular or arcuate features 150 to provide a gripping and captivation mechanism on the anchor-stop. The opposing semicircular features 150 provide a specified gripping location, which eliminates holding force variability that would be encountered due to placing the lead body 90 at different points along the jaws of the anchor-stop 120. Most preferably, the jaws cross over each other between the hinge and the arcuate sections 150, and the arcuate sections 150 each define a concave side engaging the medical lead.

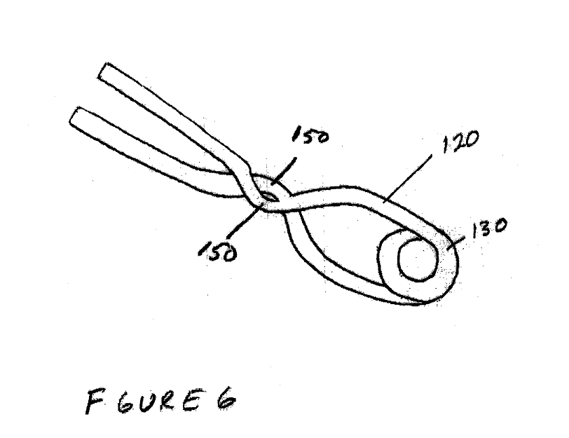

FIG. 7 shows a tissue interface feature 160 that could be built into the adjustable anchor-stop 120 to provide a soft interface to the tissue that is being captivated between the anchor stops. The surface which abuts the tissue is sized large enough to prevent the anchor-stop assembly from being drawn into the tissue; typically 0.125 inches in a gastric application. The tissue interface feature 160 is manufactured from a biocompatible material. In many applications, Silicone is the desired material due to biocompatibility and flexibility. In most applications, flexibility will be desired to avoid irritation of the tissue being stimulated or surrounding tissue.

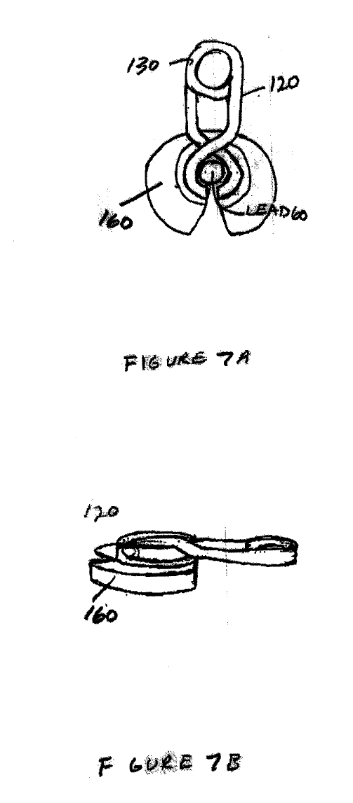

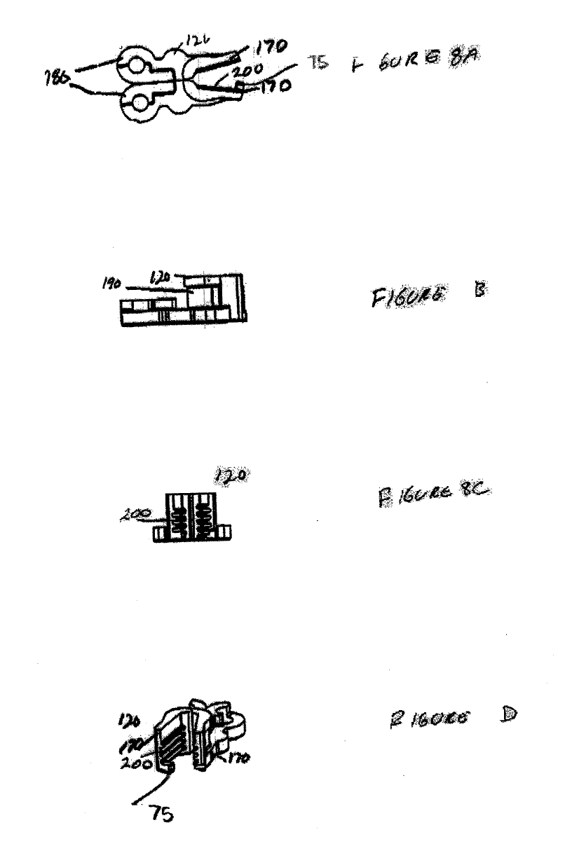

An alternative embodiment is shown in FIG. 8 and utilizes two opposing plastic pieces that have geometry to form jaws 170 for gripping the lead 60, and wings 180 to permit opening of the jaws 170 for placement on the lead 60 in a lateral direction. The two plastic halves 180 are held together by a nitinol retaining ring 190 that also provides the holding force for the anchor-stop. In the preferred embodiment, the adjustable anchor-stop is designed so that when it is in the open position it can fit through an 8 mm trocar to facilitate minimally invasive lead placement.

The anchor-stop jaws 170 contain a lead gripping feature 200 consisting of interlocking teeth or grooves that are positioned on flat planes of the opposing jaws 170 to prevent the anchor-stop from moving along the lead body 90 once the anchor has been closed. In the preferred embodiment, the plastic jaws 170 of the anchor stop are made from a biocompatible structural plastic such as polysulfone.

The jaws 170 of this embodiment have a coopeable alignment mechanism or means for aligning the free ends of each jaws relative to one another when the grip element is in the closed position, thereby tending to prevent skewing of the jaws when the jaws are in the closed position. For example, a tooth 175 may be provided on the free end of one of the jaws for engagement with a notch, cut-out, side surface or other feature of the other jaw. Such complementary interlocking structures are brought into interlocking relationship when the jaws are brought to the closed position. Further examples of complementary interlocking structures include complementary projecting and recessed portions on each jaw such that the projecting and recessed portions of one jaw define an opposite or negative structure compared to the projecting and recessed portions of the other jaw.

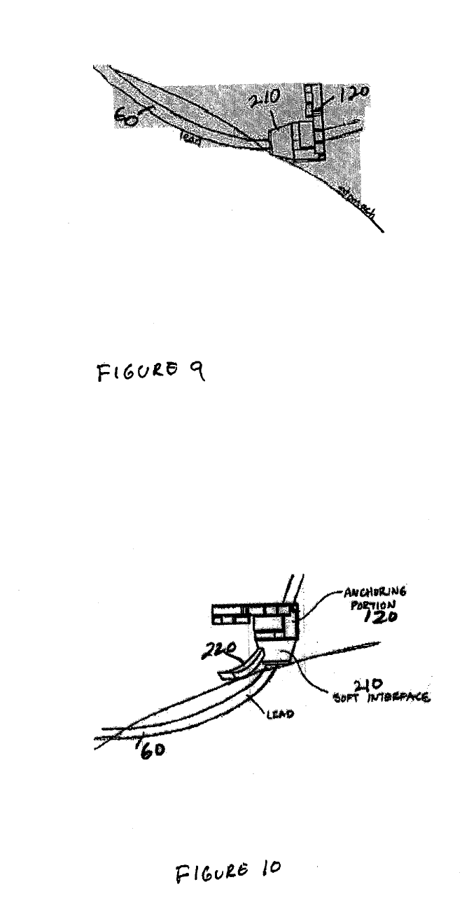

A silicone molded feature 210 (e.g., FIGS. 9 and 10) could be added to the top of the adjustable anchor-stop 120 to provide a soft interface with the body tissue. Additionally, the soft interface 210 could be shaped to provide anchor placement at a defined angle. Alternatively, the soft interface could contain wings 220 that would allow the anchor-stop to rest against the tissue while providing strain relief to prevent the anchor-stop from being drawn into the tissue. The wings 220 can also contain holes to allow supplemental fixation with sutures. The wings 220 preferably flex to allow insertion through a cannula.

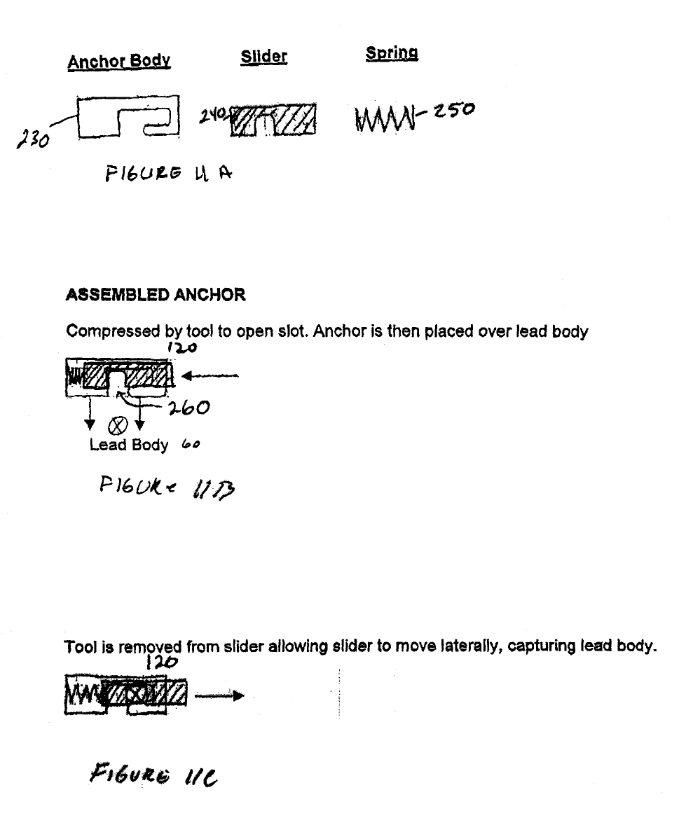

FIG. 11 shows an alternative adjustable anchor-stop 120 that is formed from an anchor body 230, a slider 240 and a spring 250. A tool is used to push the slider and compress the spring 250 which accesses a slot or opening 260 in the anchor. The anchor 120 is placed over the lead 60 via the slot 260. Once the anchor-stop 120 is in the desired position on the lead body 60, the tool is removed from the anchor 120, which allows the spring 250 to extend, forcing the slider 240 to move relative to the anchor body 230 and thereby closing the opening 260 that was used to place the anchor on the lead 60, and capturing the lead 60. The anchor body 230 and slider 240 are manufactured from a rigid biocompatible material such as polysulfone. The spring 250 is manufactured from a biocompatible spring metal such as stainless steel or nitinol.

U.S. patent application Ser. No. 10/121,484; filed Apr. 12, 2002, on “IMPLANTABLE MEDICAL DEVICE WITH CAPTIVATION FIXATION” by Carole Tronnes, John M. Swoyer and Martin T. Gerber, discloses fixation features for, among other things, gastric leads, and is incorporated herein by reference. The fixation features include an anchor or stop that is movable along the length of the lead relative to the first anchor to capture the tissue between the anchor or stop and another anchor or stop so that the lead is retained in position. Embodiments of the anchor clip of this invention are particularly suited for use with the devices disclosed in Ser. No. 10/121,484.

Thus, embodiments of the implantable therapy stimulation lead 60 with captivation fixation are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A medical lead system comprising:
a lead having a length defining longitudinal and lateral directions and at least one electrode; and
a clip having two arms biased to a closed position clamping the lead, the arms being movable against the bias to an open position allowing the clip to be moved along the length of the lead, or the clip to be attached or removed in the lateral direction.

2. A combination of the medical lead system of claim 1 with an application tool, the application tool comprising:
an elongate shaft having proximal and distal ends, the distal end being insert-able through a cannula;
at least two jaws adjacent the distal end of the elongate shaft, the jaws including a movable jaw movable relative to the other jaw to squeeze the clip to move the arms of the clip to the open position; and
a operating mechanism operable from the proximal end to move the movable jaw.

3. An implantable therapy delivery system comprising:
an implantable therapy delivery device;
at least one elongate therapy delivery element coupled to the implantable therapy delivery device, the therapy delivery element having a length defining longitudinal and lateral directions;
an adjustable anchor coupleable to the therapy delivery element, the adjustable anchor being implantable and including,
a grip element configured to be actuated between:
an open position in which the anchor may be attached or removed from the therapy delivery element laterally, or repositioned along the length of the therapy delivery element; and
a closed position in which the grip element grips the therapy delivery element,
at least two extension elements connected to the therapy grip element, the extension element extending substantially perpendicular from the therapy delivery element, and the extension elements being configured to actuate the therapy grip element.

4. The implantable therapy delivery system of claim 3 wherein the anchor may be removed or attached at any point along the length of the therapy delivery element when the grip element is in its open position.

5. The implantable therapy delivery system of claim 3 wherein the open position includes:
a first open position in which repositioned along the length of the therapy delivery element; and
a second open position further open than the first open position in which the anchor may be attached or removed from the therapy delivery element laterally.

6. The implantable therapy delivery system of claim 5 further comprising means for indicating that the grip element is in each of its first and second open positions.

7. The implantable therapy delivery system of claim 5 in which the means for indicating that the grip element is in each of its first and second open positions comprises a tool for manipulating the anchor and indicating whether the grip element is in each of its first and second open positions.

8. The implantable therapy delivery system of claim 5 further comprising means for indicating that the grip element is in each of its first and second open positions comprises a detent for releasably retaining the grip element in either its first or second open positions.

9. The implantable therapy delivery system of claim of claim 3 wherein the grip element comprises two jaws for clamping the therapy delivery element, the jaws having roughed, toothed, grooved, or sticky surfaces for gripping the therapy delivery element.

10. The implantable therapy delivery system of claim of claim 3 wherein the grip element comprises two jaws for clamping the therapy delivery element, the jaws including interlocking teeth or grooves.

11. The implantable therapy delivery system of claim of claim 3 wherein grip element includes two jaws for clamping the therapy delivery element, the jaws each having a free end and including cooperable alignment means for aligning the free ends of each jaws relative to one another when the grip element is in the closed position, thereby tending to prevent skewing of the jaws when the jaws are in the closed position.

12. The implantable therapy delivery system of claim of claim 11 wherein the cooperable alignment means comprises complementary interlocking structures on the free ends of the jaws that are brought into interlocking relationship when the jaws are brought to the closed position.

13. The implantable therapy delivery system of claim of claim 12 wherein complementary interlocking structures comprise complementary projecting and recessed portions on each jaw such that the projecting and recessed portions of one jaw define an opposite or negative structure compared to the projecting and recessed portions of the other jaw.

14. An implantable medical lead system comprising:
at least one elongate lead having a length, opposite ends;
a first stop mounted on the lead;
a second stop mounted on the lead for movement along the length of the lead relative to the first stop to capture the tissue between the stops so that the lead is retained in position; and
a clip having two arms biased to a closed position clamping the lead to prevent the second stop from moving, the arms being movable against the bias to an open position allowing the clip to be moved along the length of the lead to release the second stop.

15. A medical lead system comprising:
a lead having a length defining longitudinal and lateral directions and at least one electrode; and
a clip formed of spring wire to have two arms biased to a closed position clamping the lead, the arms being movable against the bias to an open position allowing the clip to be moved along the length of the lead, or the clip to be attached or removed in the lateral direction, the arms being offset in the longitudinal direction relative to one another.

16. The medical lead system of claim 15 wherein the spring wire is bent to form a helical hinge section connecting the two arms and permitting movement of the arms between the open position and a closed position.

17. The medical lead system of claim 16 wherein the spring wire is bent to form an arcuate section along each arm for engaging the medical lead, the arms crossing over each other between the hinge and the arcuate sections.

18. The medical lead system of claim 17 wherein the arcuate sections each define a concave side engaging the medical lead.
